(19) United States
(12) Patent Application Publication
(10) Pub. No.: US 2023/0134325 A1
(43) Pub. Date: May 4, 2023

(54) INFECTION AND DISEASE SENSING SYSTEMS

(71) Applicant: Heba BEVAN, Bromley (GB)

(72) Inventor: Heba BEVAN, Bromley (GB)

(21) Appl. No.: 17/917,903

(22) PCT Filed: Apr. 8, 2021
(86) PCT No.: PCT/EP2021/059186

§ 371 Date: Oct. 7, 2022

(30) Foreign Application Priority Data
Apr. 8, 2020 (GB) 2005213.0
Nov. 27, 2020 (GB) 2018690.4

(51) Int. Cl. A61B 5/01; A61B 5/00; G01J 5/00; G01J 5/02; G01J 5/08

(52) U.S. Cl. CPC A61B 5/015; A61B 5/0077; A61B 5/489; G01J 5/0025; G01J 5/025; G01J 5/0859; G01J 2005/0077

(57) ABSTRACT

An infection sensing system for determining whether a human or animal user has one of a plurality of infection conditions in response to a sensed condition of the user. The system includes a remote temperature measuring subsystem comprising a first, imaging sensor to capture a first image of a body part, a thermal imaging camera to capture a thermal image of the body part, and an image rocessor to process the first image to identify when the body part is present in a field of view of the thermal imaging camera. The system is also configured to determine one or more biomarker values for one or more further characteristics of the human or animal user. machine learning classifier processes the body temperature and further characteristic(s) to identify one of the infection conditions.

### FIELD

This specification relates to systems for sensing infection or disease of the human or animal body.

### BACKGROUND

Background prior art relating to non-contact human body temperature measurement can be found in WO2016/013018, GB2571379A, WO2019/061293, KR2017/0050936, WO2014/149976, CN102663355A, WO2019/041412, and US2016/0113517.

### SUMMARY

This specification generally relates to systems for sensing infection or disease, in particular using thermal imaging to remotely measuring human or animal body temperature.

In one aspect there is described an infection or disease sensing system for determining whether a human or animal user has one of a plurality of infection or disease conditions in response to a sensed condition of the human or animal user. The conditions may, for example, distinguish between the presence and absence of general illness (e.g. in some implementations which sense body temperature), or the conditions may distinguish between presence and absence of a particular morbidity i.e. the system may determine whether the human or animal has a particular condition. Alternatively the system may distinguish between the absence of morbidity and the presence of one or more of morbidities from a set of pre-determined possible morbidities.

Some implementations of the system are particularly useful in sensing the presence of respiratory disease or heart disease e.g. for determining whether the user has a particular respiratory condition, such as a coronavirus disease.

The infection or disease sensing system may comprise a remote temperature measuring subsystem for remote body temperature measurement of a human or animal user.

The subsystem may comprise a first imaging sensor to capture a first image of a body part of the human or animal user, and a thermal imaging camera to capture a thermal image of the body part. The first imaging sensor and the thermal imaging camera may have overlapping fields of view, e.g. each may have a field of view which includes the body part, when the body part is viewed by the other. A first image processor is configured to process the first image to identify when the body part is present in a field of view of the thermal imaging camera.

The infection or disease sensing system may comprise a thermal image processing subsystem to process the thermal image to identify one or more blood vessels, e.g. arteries, in the thermal image i.e. in the field of the thermal imaging camera e.g. by identifying locations, such as pixels, corresponding to locations of blood vessels. In implementations the remote temperature measuring subsystem is configured to determine a body temperature of the human or animal user from the thermal image of the blood vessels, i.e. from a part of the thermal image which includes the blood vessels. In implementations the body temperature is determined from the thermal image of the blood vessels, i.e. from locations of blood vessels in the thermal image. In implementations the body temperature is determined in response to the identification of when the body part is present in the field of the thermal imaging camera.

The infection or disease sensing system may be further configured to determine one or more biomarker values for one or more further characteristics of the human or animal user.

The infection or disease sensing system may include a classifier, configured to process the body temperature of the human or animal user determined from the thermal image of the blood vessels and the value of the one or more further characteristics, i.e. the one or more biomarker values, and to provide a classification output for selecting one of the plurality of infection or disease conditions to assign to the human or animal user.

The classification output may be a hard decision e.g. defining which of the plurality of infection or disease conditions it is most likely that the user has, or it may e.g. comprise a set of scores, one for each condition, defining a probability of the respective condition. Such scores may be used to determine one of the conditions e.g. according to a probability threshold. In particular, but not necessarily, when there are two conditions the threshold may be determined to trade true vs false positives (or negatives) e.g. based on an ROC (receiver operating characteristic) or precision-recall curve.

In some implementations the classifier operates by combining multiple biomarker values to determine a biomarker value profile for the user, which may then be processed to determine presence (or absence) of one or more conditions to be sensed. The processing may involve comparing the biomarker value profile with the profile of the condition(s), or such a comparison may be made implicitly e.g. using a trained machine learning system such as a neural network or other machine learning system. In some implementations determining the biomarker value profile for the user may involve processing the multiple biomarker values using a trained machine learning system such as a neural network.

In general the machine learning systems described in this specification may be trained conventionally, i.e. using labelled training examples obtained from some “training” users known to have the condition(s) and some users known not to have the conditions. Biomarker values obtained from such users are processed using the system and parameters of the machine learning component, e.g. weights of a neural network, are adjusted to optimise an objective function e.g. dependent upon whether a correct infection or disease condition has been assigned to a “training” user.

There is also provided a corresponding method of sensing infection or disease; and software to implement the method.

There is further provided a face mask for use with the system. The face mask comprises one or both of mask a removable microphone and a removable gas e.g. nitric oxide sensor, such that the microphone and/or gas e.g. nitric oxide sensor can be removed and the face mask discarded. To facilitate this the face mask, in particular a disposable part of the face mask, may include a filter over the removable gas e.g. nitric oxide sensor to allow air to flow through to gas e.g. nitric oxide sensor. This facilitates re-use of the gas e.g. nitric oxide sensor.

In another aspect there is described a system for remote body temperature measurement of a person or animal. The system may comprise a first imaging sensor to capture a first image of a body part of the person or animal. The system may comprise a thermal imaging camera to capture a thermal image of the body part. The first imaging sensor and the thermal imaging camera may each have a field of view which includes the body part e.g. they may have overlapping fields of view.

The system may comprise a first image processor to process the first image to identify when the body part is present in a field of view of the thermal imaging camera. The system may comprise a thermal image processor to process the thermal image to identify one or more blood vessels in the field of the thermal imaging camera. The system, e.g. the thermal image processor, may determine a body temperature of the person or animal from the thermal image of the blood vessels. The body temperature may be determined in response to the identification of when the body part is present in a field of the thermal imaging camera.

Thus the first imaging sensor may detect presence of the body part, and optionally its location within a field of field of the first imaging sensor, and the thermal imaging camera is then used to determine the body temperature from the thermal image, in particular from arteries or veins within the thermal image.

The first imaging sensor may comprise a visual camera and the first image may be a visual image. Also or instead first imaging sensor may comprise a LIDAR (e.g. time-of-flight) sensor and the first image may comprise a LIDAR e.g. 3D image. In some implementations the first imaging sensor e.g. the visual camera, and the thermal imaging camera, may be combined in a single unit.

The first image processor and the thermal image processor may be implemented as software running on a common (the same) physical processor; or distributed across processors; or may be partly or wholly in the cloud i.e. on one or more remote servers.

The fields of view first imaging sensor and of the thermal imaging camera may each have a field of view which includes the body part. In some implementations the fields of view may overlap e.g. one may be partly or wholly within the other; or they may substantially correspond to one another. In other implementations they may view the same body part from different positions. For example one, e.g. a visual camera, may view the wrist from above and the other, e.g. the thermal imaging device, may view the wrist from beneath. The first e.g. visual image processor may identify when the body part is present in the first image and hence may determine when the thermal imaging camera can see the body part.

The body temperature, once determined, may be stored and/or output, e.g. displayed locally or remotely; and/or an alert may be generated is the body temperature is greater than a threshold.

In some implementations of the system the body part is the wrist (or the equivalent in an animal). This can facilitate the thermal imaging of blood vessels. In some other implementations of the system the body part is the head. In principle the system may be configured to identify more than one body part.

The first image processor may be configured to process the first image to identify when exposed skin of the body part is present in the field of the thermal imaging camera. For example in the case of a wrist the thermal imaging, or thermal image processing, may only be triggered when clothing does not obscure the target area i.e. the blood vessels to be imaged.

In some implementations the one or more blood vessels comprise one or more blood vessels between the radius and ulna. Thus the blood vessels may but need not comprise the radial artery and/or ulnar artery (which are near the bone).

The system may be combined with a radio frequency card or token reader such as an RFID (RF Identification) or NFC (Near-Field Communication) reader for a contactless payment card, access control card or ticket, key fob, or other token; or with an optical e.g. QR code reader. The visual camera and the thermal imaging camera may then be located adjacent the card or token reader such that when the person's hand holds the card or token their wrist is located in the overlapping fields of view. For example, the reader may be located on a surface and visual and thermal cameras may be provided with a common window through the surface, closer to the reader, so that when holding the card or token the wrist is above the window.

In some implementations the thermal image processor is further configured to process the thermal image to identify a pattern of blood vessels and/or bones in the wrist. This pattern may then be used to determine an identifier for the person. This has separate utility and may be performed without determining a body temperature. The identifier e.g. a numeric or alphanumeric string, may not convey an actual identity of the person without additional information such as a link from this to a name. The identifier may be stored or output in combination with the body temperature.

Where a person is monitored on a succession of occasions, e.g. on entry to a building or place of work, an identifier for the person may be used to track changes in body temperature and to generate an alert in response to a rising temperature or in response to a body temperature elevated above an average for that person. Such an identifier may be derived from the thermal image or from a card or token as previously described.

The remote body temperature measurement system may be combined with an access control system. The access control may then be configured to restrict access to the person responsive to identification of an abnormal temperature such as a greater than a threshold body temperature, or responsive to a rising or elevated body temperature.

In principle the thermal imaging camera may be replaced by a very low-resolution or single pixel thermal sensor appropriately directed using the first, body part image.

Some implementations of the system include a microphone coupled to an audio signal processor to identify a respiratory condition, e.g. by identifying a cough, wheeze or sneeze. The system may be configured to generate an alert in response to identifying the respiratory condition in combination with an abnormal temperature. Again, if combined with an access control system the access control system may restrict access when such a combination is identified.

Features of the remote body temperature measurement system may be combined with the infection or disease sensing system described previously.

There is also provided a method of remotely measuring the body temperature of a person. The method may comprise capturing a first image of a human body part. The method may further comprise capturing a thermal image of the human body part. In implementations each image comprises a view of the body part e.g. the first image and the thermal image may overlap. The method may further comprise processing the first image to identify when the human body part is present in the thermal image, then processing the thermal image to identify one or more blood vessels in the field of the thermal imaging camera. The method may further comprise determining a body temperature of the person from the thermal image of the blood vessels.

In implementations the body part is a forearm and/or wrist.

In implementations the method includes capturing the first image and the thermal image whilst the person is using a radio frequency card or token reader such that the wrist is in a known location with respect to the radio frequency card or token reader.

In implementations the method includes using the body temperature for access control.

There is also described a system for remote temperature measurement. The system may comprise a first imaging sensor to capture a first image of a sensed area. The system may comprise a thermal imaging camera to capture a thermal image of the sensed area. The first imaging sensor and the thermal imaging camera may have overlapping fields of view.

The system may comprise a first image processor to process the first image to identify when a target is present in a field of view of the thermal imaging camera. The system may comprise a thermal image processor to process the thermal image to identify one or more regions in the field of the thermal imaging camera. The system, e.g. the thermal image processor, may also determine a temperature characterizing the target from the thermal image of the regions. The temperature may be determined in response to the identification of when the target is present in a field of the thermal imaging camera.

For example the sensed area may comprise an area of soil, and the target may comprise a structure within the soil. Such a system may be used, for example, for soil investigation on land or underwater e.g. to determine soil structure, moisture content, moisture/water location, oil and gas content, oil and gas location, and so forth.

For example, geological areas have mixtures of different substances with different densities, which heat and cool at different rates. For example there may be pockets of air, water, oil, as well as rock, sand, and so forth. A thermal image of an area captured as described above can provide a useful image of the heat absorption and hence the materials present in the ground. A thermal image of an area e.g. captured as described above, can also provide information on the moisture content of soil and foliage, e.g. where areas of plants, trees and/or soil are dry and less dry. Such an image may also be used to assess the risk of fire; e.g. where the image demonstrates an area is at a level of dryness that corresponds to an unacceptable level of fire risk and alert may be generated so that the area can be treated with water and other precautions can be taken.

One or more computer readable media may store processor control code to implement the systems and methods described above, in particular the image capture and processing and body temperature determination. The code (computer program) may be provided on a non-transitory data carrier e.g. on one or more physical data carriers such as a disk or programmed memory such as non-volatile memory (eg Flash) or read-only memory (Firmware). Code and/or data to implement examples of the system/method may comprise source, object or executable code in a conventional programming language, interpreted or compiled),such as C, or assembly code, or code for a hardware description language. The code and/or data to implement the systems may be distributed between a plurality of coupled components in communication with one another.

### DRAWINGS

These and other aspects of the invention will now be further described by way of example only, with reference to the accompanying Figures, in which:

FIG. 1 shows an example system for remote body temperature measurement of a person or animal;

FIG. 2 shows example images captured using the system of FIG. 1;

FIG. 3 shows an example process illustrating operation of the system of FIG. 1;

FIG. 4 shows a scanning version of the system of FIG. 1.

FIG. 5 shows a block diagram of an example infection or disease sensing system.

FIG. 6 shows an example thermal image from the system of FIG. 5.

FIG. 7 shows a face mark for the system of FIG. 5.

FIG. 8 shows a process of operation of the system of FIG. 5.

FIG. 9 shows a graph of nitric oxide level sensed by the system of FIG. 5.

FIG. 10 shows heart rate in beats per minute on the y-axis measured by the system of FIG. 5 and by a reference system.

FIG. 11 shows a histogram of body temperature measurements made by the system of FIG. 5.

Like elements are indicated by lie reference numerals.

### DESCRIPTION

Referring to the figures, there are first described systems for remotely measuring human, or animal, body temperature using thermal imaging.

Implementations of the device, e.g. of a system as previously described, can enable unobtrusive identification of one or more of: (i) temperature, (ii) physical appearance of the wrist, (iii) the layout of bones and veins of the wrist (unique to each person), (iv) symptoms of ill health via sound, including coughing, wheezing and sneezing, and (v) a location of the person being scanned.

The system can be connected to points of entry (doors, barriers, etc) and if a person does not pass certain pre-set criteria (e.g. a temperature below a pre-set point) an alert may be sent to those responsible for medical care and security, and a security barrier may remain closed. This can inhibit those with illnesses from coming into particular premises and from coming into proximity with others and potentially spreading their illness.

Additionally, as the wrist bone and vein structure in combination are unique to a person, this may be used for security purposes, either as a standalone or in combination with ID or a pass, to identify individuals that seek entry. Should a scan fail to meet preset requirements (e.g. does not match the bone and vein layout of authorised individuals), the barrier may remain closed and an alert may be sent e.g. to those managing security on the premises.

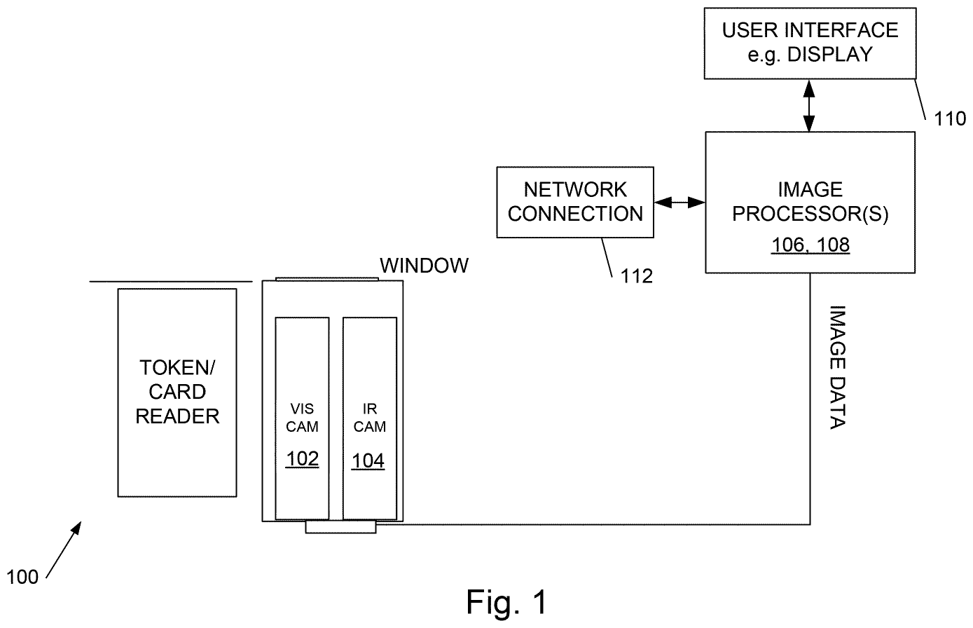

Referring to FIG. 1, an example system 100 comprises a camera 102 combined with a thermal imaging sensor 104 and uses artificial intelligence (machine learning), implemented by one or more processors 106, 108, to identify the wrist by its outline, bone structure, vein layout and temperature.

The thermal image sensor captures a thermal image and the camera captures a visual image. A processor processes one or both images to identify the image(s) as coming from the wrist.

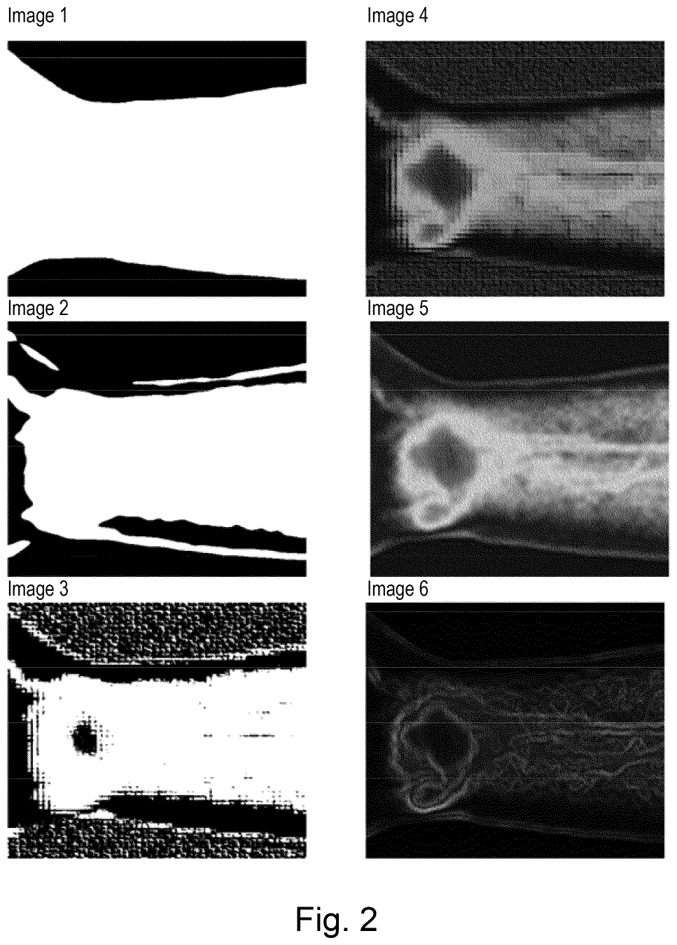

FIG. 2 shows images of the bottom of a wrist captured by the system at different distances—Images 1-6—showing how the captures image changes.

Many machine learning systems are known for identifying and/or segmenting images. Such a system may be trained to identify a body part e.g. wrist, in a visual image. A similar system may be trained to identify and locate blood vessels in a thermal image.

The device/system has an infrared camera 104 and a normal i.e. visible image camera 102; these may focus at a fixed distance, the distance to the wrist. The visual camera captures an image of the wrist (and may map the wrist). When this has been done, the thermal image determines temperature.

The sensors (cameras) may take multiple measurements e.g. at different distances, as the wrist approaches the cameras (FIG. 2). The system may then be configured to select one or more of the captured image for further processing for determining a body temperature.

All the captured data (images) is analysed and processed before a temperature record logged. The processor processes the images, ensuring that the visual image of the body part is of the wrist and comprises e.g. veins, and thus that the thermal image captures the veins and blood flow of the wrist, and therefore that the thermal sensor takes the temperature of the skin. The wrist may hover no more than 5-3 cm from the sensor (cameras). The visual camera/first image processor may be configured to identify presence of a set area between the two sides of the underside of the wrist. The closer the wrist to the sensors the more accurate the temperature reading. The two processed images are may be combined by the processor (e.g. a CPU) and mapped into a single image that includes both the visual, physical image of the wrist and the thermal image of the veins and bones.

The system may also be configured such that the visible and/or infrared camera captures images from the top side of the wrist. The system may incorporate an additional LIDAR sensor for medical purposes and/or to enhance biometric sensing of the external visual image and the vein and bone structure of the wrist.

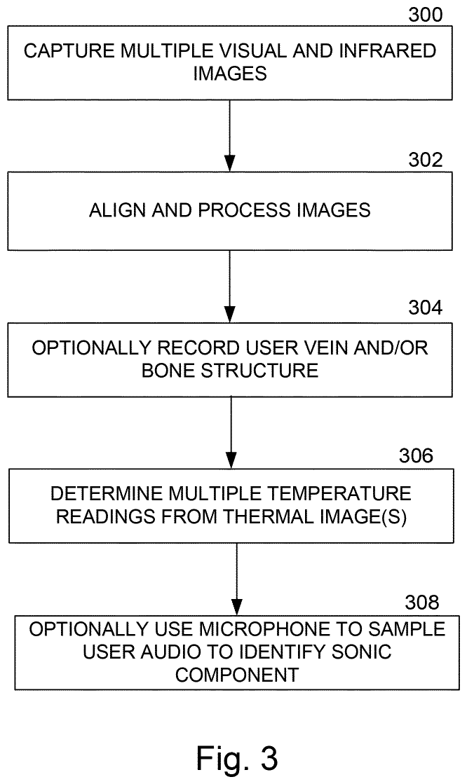

A process illustrating operation of the system is shown in FIG. 3.

A camera combined with an infrared thermal imaging sensor uses an image processor trained using machine learning to identify the wrist by its bone structure and/or the main arteries.

The infrared thermal sensor captures a thermal image and the camera captures a corresponding visible light image (step 300). Multiple images may be captured.

Visible light may comprise light with a wavelength the range 380-750 nm. The thermal imaging camera may be configured to capture electromagnetic radiation with a wavelength the range 7 or 8 microns to 14 or 15 microns.

The processor processes one or both images and identifies the image as coming from the wrist. In implementations the processor aligns the thermal image(s) and the corresponding visible light image(s) (step 302).

A machine learning-trained module, e.g. a trained neural network, identifies e.g. one of the main arteries in the wrist and the infrared sensor/processor determines the temperature of the wrist. Optionally the processor records the (unique) bone and/or vein structure of the person (step 304).

In implementations the image sensors are configured to “listen” for an image, the result is recorded, the images are compared/combined, and analysed by the processor, which generates an image processing result and an optional alert.

The infrared thermal sensor may take multiple temperature readings e.g. at various distances as the wrist approaches the device, e.g. utilising the vein/bone structure to select where to record temperature (step 306). Image capture may comprise, e.g. capture of a snapshot of the region between the radius and ulna bones highlighting and identifying the positions of the arteries/veins. Once identified, the thermal image can be processed to takes a temperature of the skin; the accuracy can be as good as approximately 0.02 C.

The system may have a microphone to sample audio from the person e.g. to capture actions of/by the person that have a sonic component (step 308). For example the microphone may capture and analyse coughs, sneezes and wheezing. Audio from the microphone may also be processed, if desired, to approximately localise a position (or direction) of the person.

Machine learning may be used to train an audio processing system, e.g. a trained neural network, to sample the captured audio to identify a respiratory sound e.g. continuous coughing or wheezing. The audio detection may be localised to the person who is the source of the coughs, sneezing or wheezing by measuring the amplitude or energy of the captured sound.

Applications for the technology described herein include entrances, card readers, security biometrics, blood flow identification and monitoring of a live human or animal, blood flow speed and volume monitoring to assess circulatory status and health.

A set of sensors/systems as described above can be used to wirelessly scan individuals for e.g. COVID-19 symptoms as they approach entrance points of buildings. The system may include an alert system that relays information in real time, enabling symptom positive individuals to receive care immediately and preventing them from coming into close proximity with others. Deployment sites may include hospitals, pharmacies, workplaces and train stations. Such a system may also be used for security as wrist vein size and layout are unique to an individual.

The device may be used to detect flu, cold, bronchitis, asthma symptoms, and respiratory illness in general.

A system as described herein can be integrated with sensors at doors, turnstiles, and barriers restricting entry to buildings, as illustrated in FIG. 1.

In some implementations, for the door, turnstile or barrier to open, an individual scans their wrist. If the scan does not meet set requirements of temperature and/or vein layout (personal identity), the door, turnstile or barrier will not open. The system can record and register those that pass through the door, turnstile or barrier, on entry and/or exit, and may send an alert via wireless or wired internet where an individual does not meet preset conditions, e.g. that the individual does not have a fever, and medical assistance can be provided if needed.

As shown in FIG. 1, the system may be physically arranged so that whilst the person is using a radio frequency card or token reader such that the wrist is in a known location with respect to the radio frequency card or token reader. For example, the first imaging sensor and the thermal imaging camera have overlapping fields of view, and the visual camera and the thermal imaging camera are located adjacent a card or token reader such that when the person's hand holds the card or token their wrist is located in the overlapping fields of view. In this way a “fever scan” may be performed without asking the user to perform any additional actions, simplifying use of the system and improving behavioural compliance: whilst the reader is arranged so that in swiping an access control device the user's wrist/forearm passes over the remote temperature measurement system. Optionally one or more physical constraints may be included to inhibit access to the reader except via/over the temperature measuring system, but often such constraints are not needed.

If the microphone is present and detects symptoms of coughing, sneezing or wheezing and the symptoms do not meet one or more predetermined requirements, e.g. frequency of coughing (or alternatively do meet such a requirement, depending on how the requirement is defined), the system may send an alert via wireless or wired internet and may disable or not enable entry through the door, turnstile or barrier. This audio sensing system may be combined with the remote temperature measurement system so that e.g. both body temperature and captured audio must be within tolerance to allow access.

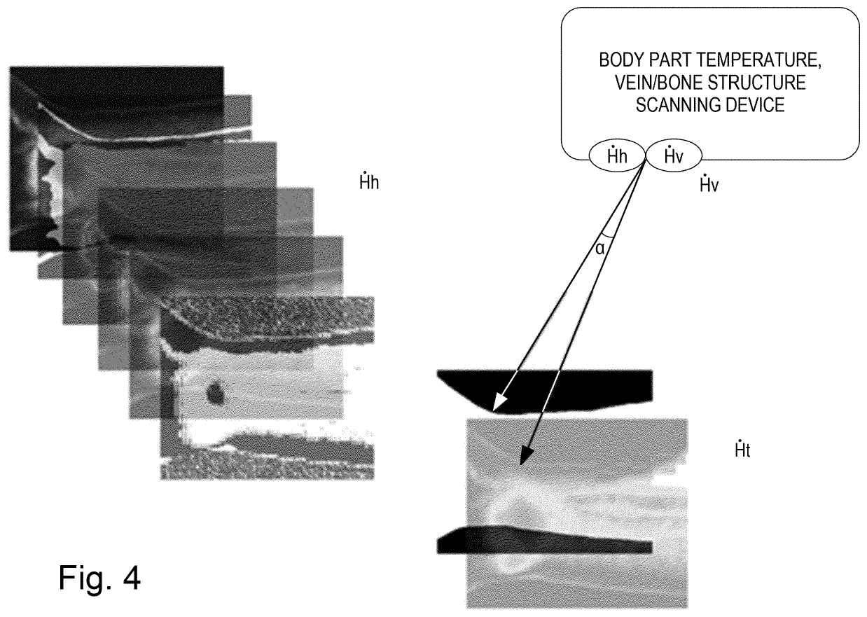

As illustrated in FIG. 4 the visual or thermal imager(s) may scan the body part e.g. wrist, e.g. in x, y and/or x-directions, to provide an image, rather than e.g. capturing an image frame in a single exposure. In FIG. 4 \dot{H}h represents frames (xh, yh, zh) from the thermal imager captured from the target body part over a scanning period of time with a scan angle (in time) α; \dot{H}v represents frames (xv, yv, zv) from the visual camera captured from the body part over the period of time; and \dot{H}t represents aligned frames (xt, yt, zt) of the target body part which has been mapped over the period of time. The processor combines \dot{H}h and \dot{H}v to determine a temperature of the body part, and optionally to identify locations of veins, and the bone structure.

### Infection and Disease Sensing

There is now described an infection or disease sensing system, which may use an remote body temperature measurement system as previously described.

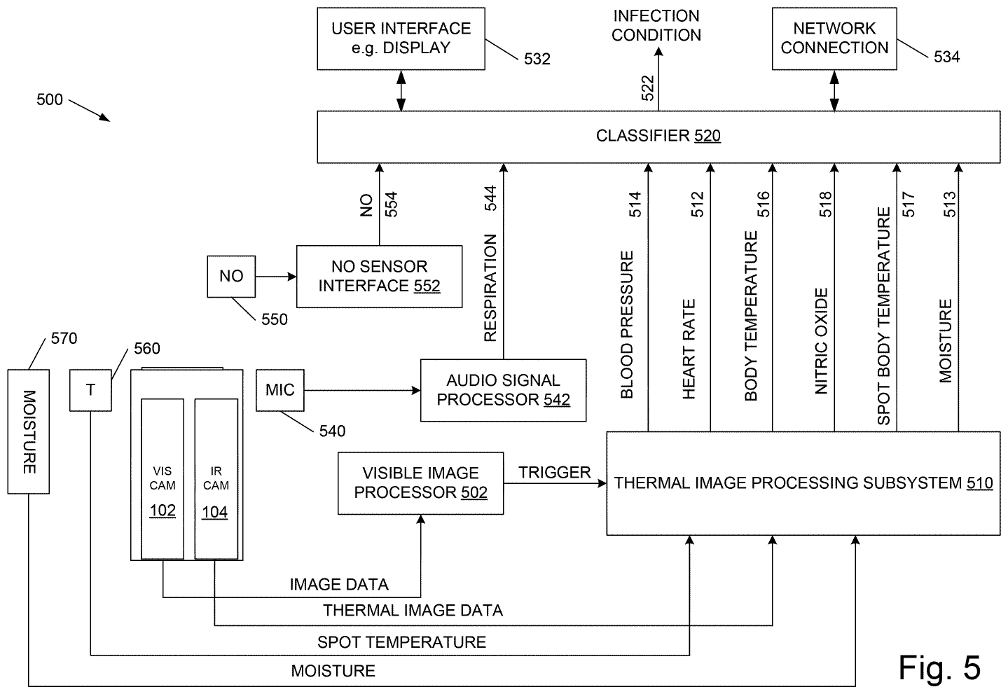

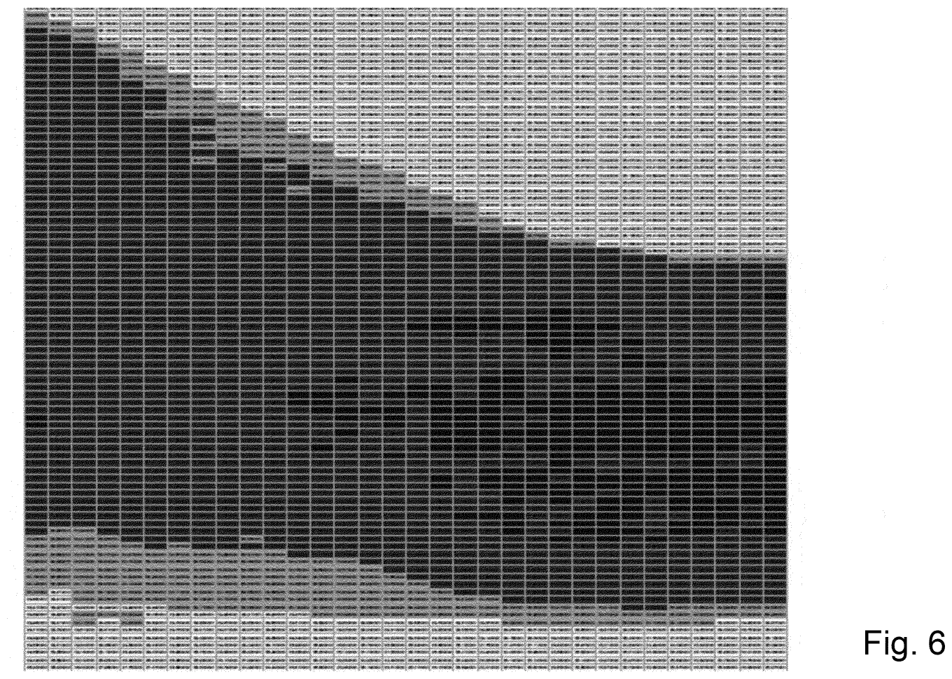

FIG. 5 shows a block diagram an example infection or disease sensing system 500. The system includes a remote temperature measuring subsystem comprising a visible image camera 102 coupled to a visible image processor 502. The remote temperature measuring subsystem also includes an infrared i.e. thermal imaging camera 104, for example operating in the 8-14 μm band. In some implementations the thermal imaging camera has an output which, for each of a plurality of pixels of a thermal image, provides a corresponding temperature of an imaged object e.g. measuring to 0.1° C. or 0.01° C. Such thermal imaging cameras/systems are commercially available devices. An example thermal image of part of a forearm is shown in FIG. 6 (the pixels contain individual temperature measurements, although too small to read in the figure).

In implementations the visible image processor 502 is configured to identify when a body part such as a wrist or forearm is present in the image, more particularly when the body part is present within a defined physical location in relation to a field of view of the thermal imaging camera. This may correspond to a lateral position within the field of view and/or a distance from the thermal imaging camera to ensure that the body part occupies a sufficient proportion of the field of view and/or is in focus. When used with an animal the body part may instead comprise part of a leg of the animal, or another body part. Any suitable image processing/image recognition techniques may be employed e.g. machine learning based techniques.

In some implementations the system may include one or more distance sensors (not shown in FIG. 5) such as an optical (laser) or RF distance sensor, to sense a distance of the body part, e.g. arm/hand, from e.g. the thermal imaging camera. This may be used to provide distance feedback to the user e.g. as described later, to facilitate the user moving their body part e.g. arm/hand, to a correct sensing location.

The thermal imaging camera 104 is coupled to a thermal image processing subsystem 510 to process one or more thermal images from the camera. The thermal image processing subsystem 510 may be coupled to the visible image processor 502 to trigger capture and processing of thermal images when the body part is determined, by the visible image processor 502, to be in position within the thermal imaging camera field of view.

The thermal image processing subsystem 510 may be configured to identify locations of one or more blood vessels such as arteries in the thermal image, e.g. by applying a temperature threshold. The threshold may be determined by calibration based in thermal images captured by the system. The locations of the blood vessels may be defined by those pixels having a temperature greater than the threshold. The pixels in the thermal image at the locations of the blood vessels may be used to determine the body temperature 516 of the human or animal user, e.g. by taking a mean or maximum temperature value .

In implementations the thermal image processing subsystem 510 is configured to determine one or more biomarker values for one or more further characteristics of the human or animal user from one or more of the thermal images.

The thermal image processing subsystem 510 may configured to determine a heart rate biomarker value 512 for the heart rate of the user from a time series of the thermal images. For example with a thermal imager capable of accurate temperature measurement a user heart rate may be determined from the small temperature fluctuations which are visible in the thermal image. These may be processed individually e.g. by pixel, or averaged over larger areas or over all of the image before processing. The processing may e.g. comprise determining an autocorrelation coefficient and identifying a peak (e.g. a smallest time interval peak). Where more than one heart rate is determined, e.g. from different image regions, an average may be taken. A suitable frame rate for such a time series is around 10 frames per second.

In a similar way thermal image processing subsystem 510 may configured to determine a blood pressure biomarker value 514 for the blood pressure of the user from a, e.g. the, time series of the thermal images. A value characterising or dependent upon the blood pressure may be determined from a magnitude of the temperature fluctuations which are visible in the thermal image, again optionally averaged. In implementations of the system it is not necessary to determine a physiologically exact measure of blood pressure; a value which has some dependence on blood pressure is sufficient.

The thermal image processing subsystem 510 may also or instead configured to determine a nitric oxide biomarker value 518 for a level of nitric oxide in the user from a, e.g. the, time series of the thermal images. Nitric oxide (NO) affects dilation of the blood vessels, and is apparently affected by various infections. Without wishing to be bound by theory, the nitric oxide biomarker value may be determined from a measure of an area of the body part within a temperature range. For example an upper and lower temperature threshold may be applied to pixels of the thermal image and a number of pixels having a temperature within the temperature range may be counted. Optionally an average over a local group of pixels may be taken beforehand e.g. to increase temperature resolution at the expense of spatial resolution. Optionally only a part of the captured thermal image is processed e.g. a region of the forearm.

The upper and lower temperature threshold may be determined by experiment or calibration with a particular thermal imaging camera. For example a level of NO released through the skin may be measured and the upper and lower temperature thresholds chosen so that this measurement correlates with the measured area (an exact correspondence is not required). In some implementations the temperature threshold used to identify locations of the blood vessels in the thermal image may be used as the upper temperature threshold.

In some implementations the infection or disease sensing system includes a separate gas e.g. nitric oxide sensor 550 and an NO sensor interface 552 to process a signal from the sensor to determine a second biomarker value 554 for a level of gas e.g. nitric oxide in the user. This may depend upon a level of gas e.g. nitric oxide leaving the body through the skin of the user. Without wishing to be bound by theory it is believed that the level of NO measured externally in this way corresponds to a level of NO within the user's body, though again an exact correspondence is not required. In some implementations the NO sensor 550 may be incorporated into a face mask, as described below.

Those implementations of the infection or disease sensing system which determine a nitric oxide level biomarker may use the thermal image, an NO sensor, or both.

Also or instead of sensing NO the system may include a gas sensor to sense a level of oxygen and/or carbon dioxide in the vicinity of the user and to determine a corresponding biomarker value for use by the classifier.

In general the second biomarker value may represent for a level of any gas in the user, for example one or more of nitric oxide, oxygen, carbon dioxide, methane, or ammonia. Thus the system may include one or more gas sensors to sense a level of one or more of these gases in or from the user. The infection or disease sensing system may be configured to determine the second biomarker value, e.g. by processing a signal from the gas sensor(s).

In some implementations the system has a housing which has a generally C-shaped vertical cross-section; the housing may extend longitudinally to define an elongated C-shaped aperture. When, in use, a user places their arm or wrist within the opening of the “C” this effectively defines a chamber within which gas may be sensed. In some implementations the gas sensor is directed downwards from an upper part of the C, to inhibit dust ingress. Although described as C-shaped, in practice the sides of the C may be generally flat. For example the housing may have an upper part, a lower part and a side wall. The upper and/or lower part may house the camera and thermal imaging sensor.

In some implementations the infection or disease sensing system includes a microphone 540 and coupled to an audio signal processor 542 to process a signal from the microphone to determine an audio biomarker value 544 for a respiratory infection or respiratory disease in the user. The audio signal processor 542 may comprise a machine learning model such as a neural network, trained to identify, in captured audio from the microphone, one or more sounds characteristic of a respiratory infection or respiratory disease. Such sounds may include e.g. a cough characteristic of a coronavirus infection, or breathing or speech having a wheezing character characteristic of asthma. The audio signal processor 542 may be trained in a conventional manner using supervised training based on a corpus of labelled training examples. In some other implementations the audio signal processor 542 may be configured to identify a cough (with or without machine learning), and to determine a frequency of coughing (e.g. how often a cough is detected or how many coughs are detect in a time interval). The audio biomarker value 544 may be dependent upon the determined frequency of coughing. The audio biomarker value 544 may be a scalar value or a vector e.g. a feature vector, which may be derived from a layer below an output, classification layer of the audio signal processor 542. In some implementations the microphone 540 may be incorporated into a face mask, as described below.

In some implementations the infection or disease sensing system includes a spot or point temperature sensor 560 to remotely sense a temperature at a spot or point location on the body part to determine a biomarker value 517 for a point temperature at a target location on the surface of the body part. The spot or point temperature sensor 560 may comprise a remote e.g. optical temperature sensor such as an infrared thermometer or pyrometer. This can provide an accurate point temperature reading. For example the target location on the body part may be a point on or between the radial and ulnar arteries in the wrist.

In some implementations the image from the visual camera 102 may be used to provide feedback to the user so that they are able to adjust a position of their arm to align the point sensed by the point temperature sensor 560 with the target location. For example a user interface 532 of the system may have a display which indicates a direction to move for alignment, and when correct alignment is reached. For example this may be achieved with a bar which moves with the user's body part, the object being to move the bar into a green region. Lateral position and/or depth (z-direction position) may be sensed and fed back. In some variants an additional sensor is used instead of the visual camera 102. Optionally user feedback of this type may also be provided to allow the user to move their body art into alignment with the thermal imaging camera, though this is less important because of the field of the thermal imaging camera.

An example system may be combined with an RFID card/tag reader e.g. on an upper surface of the system housing. A screen may be provided to show the position of the user's wrist and forearm. In one implementation, by moving their arm the position of a line in a bar on the left of the screen is moved from a red region to a green region. After a reading has been taken e.g. a set of thermal images captured, an indicator e.g. lights to either side of the screen, changes from blue to green and a tick appears on the screen, whereupon the user can remove their arm.

Some implementations of the infection or disease sensing system include a moisture sensor 570 to remotely sense moisture e.g. sweat, on a surface of the body part to determine a moisture biomarker value 513 for a level of sweat on the surface of the body part. In some implementations the moisture sensor comprises an optical reflectivity sensor to remotely sense moisture on the surface of the body part. In some other implementations the moisture sensor comprises an RF sensor which may e.g. operate similarly.

Some implementations of the infection or disease sensing system include a humidity and/or temperature sensor (not shown in FIG. 5) to sense local humidity and/or local temperature i.e. in the vicinity of the user/body part. The sensed humidity level and/or local temperature level may provide one or more additional inputs to the classifier. This is useful because some of the sensed parameters, such as skin surface temperature, can depend on local humidity and/or temperature. Thus by including such data as a parameter input to the classifier the classifier can learn to compensate for local humidity and/or temperature effects on the sensed biomarker values. Local humidity and temperature may be measured in many ways. In one approach an RF humidity sensor is used to measure local humidity.

Some implementations of the infection or disease sensing system include an SpO₂ (blood oxygen saturation) sensor; this may be suitable for remote reading so that a physical clip on the user's finger is not needed. The sensed blood oxygen saturation may provide a further input to the classifier.

In principle other user-derived/user-characterizing data may be provided to the classifier, for example blood type data. The user may input such data via an input device such as a keyboard.

In implementations the biomarker values are provided to a classifier 520 e.g. a trained neural network. The classifier 520 has an output 522 which indicates an infection or disease condition. The infection or disease condition may be one of a predetermined plurality of possible infection or disease conditions.

The classifier outputs may define infection and/or disease conditions e.g. comprising one or more of: no detected infection, an infection e.g. an infection (such as a coronavirus infection or COVID), heart disease, asthma, diabetes, and an inflammatory infection. The classifier may have one output per condition or class/category into which the user, more particularly the user's sensed data, is categorised. In some implementations the classifier may provide a simple no infection/infection output i.e. there may be just two outputs or classes; in some other implementations the classifier may similarly provide just two outputs e.g. no disease/disease where the “disease” may be of a particular type e.g. heart disease. In some other implementations the classifier may provide three or more outputs corresponding to one of e.g. no infection, infection, and disease (such as cardiovascular disease, heart disease, asthma, or other respiratory disease such as bronchitis); or to no infection, infection type 1, and infection type 2; or to no disease, disease type 1, and disease type 2; and so forth.

The output 522 may comprise e.g. an indication of one or the possible infection or disease conditions and/or an indication of a respective probability of each condition. Optionally the system may include provision for a sensitivity-specificity trade-off to be set e.g. by an operator, e.g. based on a system calibration to determine an ROC or precision-recall curve.

The output 522 may be provided in any suitable manner e.g. on a display on the device, or as a hard copy, or over a network, or stored in memory. In some implementations a display on the device is configured to display an optical code, e.g. a QR code, which includes the sensed parameters (levels of the sensed biomarkers), and the infection condition, and optionally user-entered data; optionally an identifier of the particular scan may also be included.

The classifier may be implemented as a neural network e.g. having an input layer to receive a feature vector comprising values e.g. normalized values, of the biomarkers. The neural network may then comprise one or more neural network layers coupled to the input layer e.g. one or more fully-connected neural network layers and/or one or move convolutional neural network layers. These may be followed by an output neural network layer e.g. fully connected layer, which may be followed by e.g. a softmax function to convert output values such as logits to probability values associated with the possible outputs. For example each output may be associated with a respective classification category i.e. one of the infection or disease conditions. In other implementations the classifier may be configured to implement another machine learning technique such as a support vector machine or a random forest.

Information derived from the infection or disease condition output 522 may be e.g. displayed to the user and/or to an operator; and/or stored to later access, transmitted to a remote location, used for user access-control, or in any other way.

The classifier 520 may be trained in a conventional manner using supervised training based on a corpus of labelled training examples. For example to identify one or more infections or diseases a training set of users is identified each having either no infection or disease or one of the one or more target classifications. These users are then presented to the system, to provide a labelled data set comprising for each user an input feature vector and a correct classification category output. Optionally this may be done under a range of conditions such as different local temperatures and/or humidity values. No individual user identification is needed for this. Techniques such as regularization may be used to reduce overfitting if the data set is small; known techniques such as class weighting or oversampling can be used to reduce effects due to class imbalance; or the training data set may be constructed so that there are balanced numbers of training examples in each classifier category.

In some implementations a relatively small training dataset may be used for initial training, and then the system may improve its performance during use. Specifically input feature vector data may be collected during use of the system together with a (potentially anonymous) user identifier. Then where it is later independently established that a particular uses has or does not have a condition associated with one of the output classifications (categories), this information may be used for further training. Optionally multiple different systems may share training data.

The infection or disease sensing system may also include non-volatile storage (not shown) and/or a network connection 534 for a wired and/or wireless connection to e.g. a remote server. These may be used e.g. to store and/or transmit information derived from the infection or disease condition output 522, and/or a user ID, and/or any of the information from which the output 522 was derived e.g. one or more biomarker values.

As previously described, the infection or disease sensing system may include a user interface 532, e.g. a screen. This may be used to identify the user i.e. to input user identity data for determining a user ID, which may comprise a numeric and/or alphabetic string. The user interface 532 may include a keypad and/or it may include and RFID or other contactless technology reader to read the user identification data from a user identification device such as an RFID tag or NFC (near-field coupling) ID card. In some implementations the system may include a biometric identification system to identify the user; and/or the pattern of blood vessels may be used to identify the user.

The infection or disease sensing system may configured to determine, for storage and/or transmission, a cryptographically protected combination of the user ID and one or more of: the body temperature of the user, the one or more further characteristics e.g. one or more of the biomarker values, and data from the classification output.

In some implementations the cryptographically protected combination comprises a blockchain to link the user ID with a timestamped block comprising the one or more of: the body temperature of the user, the one or more further characteristics, and the data from the classification output. Such a block may include the user ID. This may be used e.g. to provide a chain of successive timestamped recordings of a user's infection or disease status.

The invention also contemplates that such a blockchain based approach may be used with an infection or disease sensing system which omits one or more of the features described above e.g. the visual camera 102 or thermal imaging camera 104—applications of this approach are broad and not limited to the specific system described but may be used with any system which measures one or more characteristics of a user, determines an infection or disease status e.g. an infection or disease condition as described above, and combines this information with an identifier of the user, e.g. to record successive infection or disease check events using successive blocks of a blockchain.

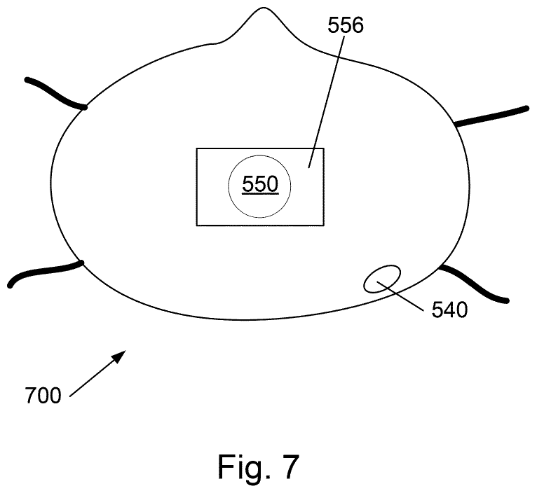

As shown in FIG. 7, the microphone 540 and nitric oxide sensor 550 may, in some implementations be provided in a disposable face mask 700. The microphone 540 and nitric oxide sensor 550 may therefore be removable from the face mask. The microphone 540 may be on an outer surface of the mask, and detachable. The nitric oxide sensor 550 may be mounted on a protective, disposable filter 556, to allow air from the user to reach the sensor whilst protecting the sensor.

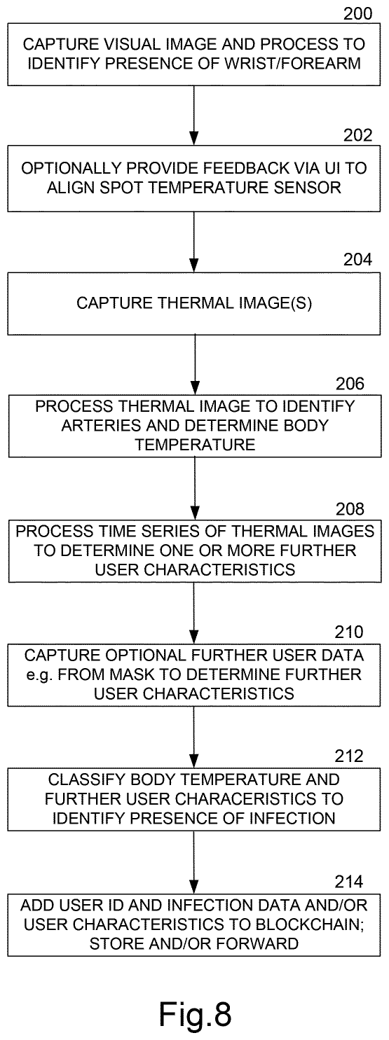

FIG. 8 shows an example process, which may be implemented by software controlling the infection or disease sensing system 500, to sense user infection or disease. Many of the steps of FIG. 8 may be performed in a different order to that shown.

At step 200 the system captures a visual image using camera 102 and processes this to identify presence of e.g. a user wrist/forearm. The system may optionally provide feedback, e.g. via user interface 532, to assist the user in aligning the point temperature sensor, if present (step 202). The system then captures one or more thermal images (step 204).

The thermal image(s) are processed to identify the location of blood vessels e.g. arteries, and these are then use to determine a body temperature for the user (step 206). Where a time series of thermal images has been captured these may be processed to determine one or more further characteristics, e.g. heart rate, blood pressure, or nitric oxide level, as described above (step 208). The system may optionally capture further user data for determining further user characteristics e.g. from a face mask and/or other sensor(s), also as described above (step 210).

The system then processes the body temperature determined for the user and any further user characteristics determined by the system using classifier 520 to identify the presence of infection or disease (step 212). This may be a binary output e.g. yes/no to the presence of infection or disease, and/or may indicate more information such as a type of infection or disease or a probability of infection or disease/absence of infection or disease.

The system may also store or transmit a result of the infection or disease sensing, optionally with some or all of the data on which the result was based, e.g. in a cryptographically secure manner, e.g. by adding the result and a user ID to a blockchain (step 214).

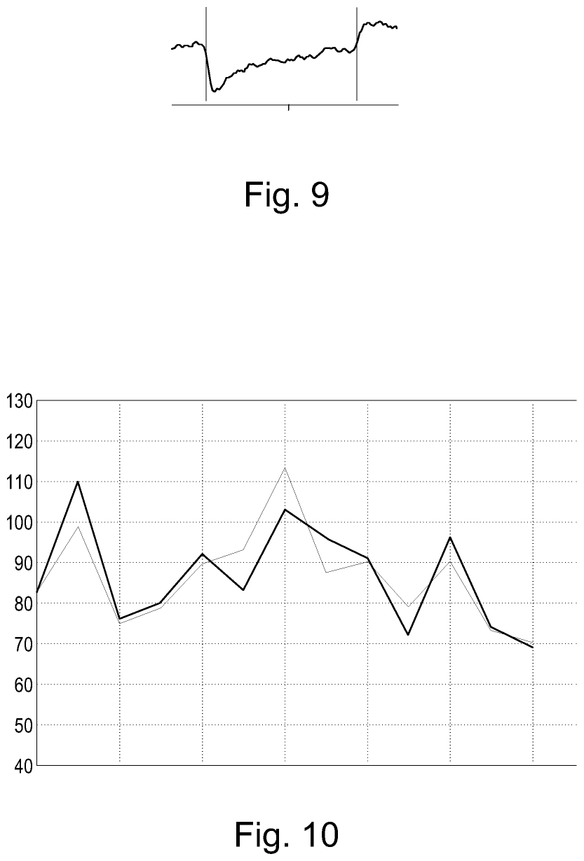

FIG. 9 shows an example of a level of nitric oxide sensed by an implementation of the system of FIG. 5. The first and second vertical lines indicate, respectively, where the user's forearm was inserted into and removed from the chamber defined by the C-shaped housing. The dip in the curve indicates an increase in sensed NO level.

FIG. 10 shows heart rate in beats per minute sensed by system on the y-axis with, for comparison, a second curve showing heart rate measured by a reference system. The different heart rate samples are distributed along the x-axis; the bold curve is the reference.

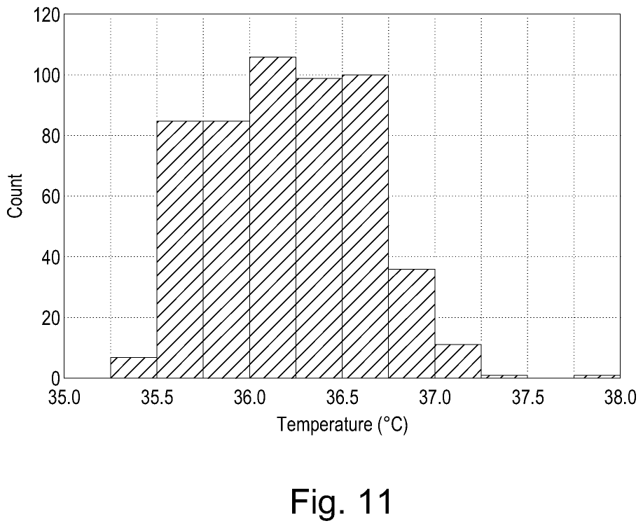

FIG. 11 shows a histogram of body temperature measurements made by the system, indicating that accurate temperature determinations are possible. The system combines these with the other sensed parameter(s) to sense infection, for example due to a coronavirus or other condition.

One example implementation of the system scans the wrist and accurately measures multiple variables, including one or more of: gas emissions, blood oxygen level, blood flow, heart rate, frequency of cough and temperature. The measurements are amalgamated using artificial intelligence (a machine learning process) to build an overall measurement profile that may then be compared against a multi-variable profile of a condition to be sensed e.g. COVID-19.

Users may then receive one of three clear results: “Success” when their measurement profile does not match that of the target condition e.g. COVID-19, “Re-scan” when the user needs to re-position their wrist into the correct position for scanning, and “Do not proceed—seek medical advice” when their measurement profile matches that of the target condition e.g. COVID-19.

Some implementations of the system can produce a result within 5-45 seconds. The system can be physically small and can be deployed at the entrance of a building or property to rapidly scan large numbers of people, enabling those with a profile matching that of e.g. COVID-19 to be quickly removed from the area to seek medical attention and confirmatory testing. Some implementations of the system may continue to learn after deployment e.g. a machine learning component of the system may continue to be trained based on test results.

Features of the method and system which have been described or depicted herein in combination e.g. in an embodiment, may be implemented separately or in sub-combinations. Features from different embodiments may be combined. Thus each feature disclosed or illustrated in the present specification may be incorporated in the invention, whether alone or in any appropriate combination with any other feature disclosed or illustrated herein. Method steps should not be taken as requiring a particular order e.g. that in which they are described or depicted, unless this is specifically stated. A system may be configured to perform a task by providing processor control code and/or dedicated or programmed hardware e.g. electronic circuitry to implement the task.

Aspects of the method and system have been described in terms of embodiments but these embodiments are illustrative only and the claims are not limited to those embodiments. Those skilled in the art will be able to make modifications and identify alternatives in view of the disclosure which are contemplated as falling within the scope of the claims.

1. An infection or disease sensing system for determining whether a human or animal user has one of a plurality of infection or disease conditions in response to a sensed condition of the human or animal user, the infection or disease sensing system comprising:
a remote temperature measuring subsystem for remote body temperature measurement of a human or animal user, the subsystem optionally comprising:
a first imaging sensor to capture a first image of a body part of the human or animal user;
a thermal imaging camera to capture a thermal image of the body part;
wherein the first imaging sensor and the thermal imaging camera have overlapping fields of view;
a first image processor to process the first image to identify when the body part is present in a field of view of the thermal imaging camera;
a thermal image processing subsystem to process the thermal image to identify locations of one or more blood vessels in the thermal image;
wherein the remote temperature measuring subsystem is configured to determine a body temperature of the human or animal user from the thermal image of the blood vessels; and
wherein the body temperature is determined from the thermal image of the blood vessels in response to the identification of when the body part is present in the field of the thermal imaging camera;
wherein the infection or disease sensing system is further configured to determine one or more biomarker values for one or more further characteristics of the human or animal user; and
a classifier, configured to process the body temperature of the human or animal user determined from the thermal image of the blood vessels and the one or more biomarker values, to provide a classification output for selecting one of the plurality of infection or disease conditions to assign to the human or animal user.

2. The system of claim 1 wherein the thermal image processing subsystem is configured to determine one or more of the biomarker values from one or more of the thermal images, and wherein the one or more biomarker values include the one or more biomarker values from one or more of the thermal images.

3. The system of claim 2 wherein the thermal image processing subsystem is configured to capture a time series of the thermal images, and to determine a biomarker value for the heart rate of the user from the time series of the thermal images.

4. The system of claim 2, wherein the thermal image processing subsystem is configured to capture a time series of the thermal images, and to determine a biomarker value for the blood pressure of the user from the time series of the thermal images.

5. The system of claim 2, wherein the one or more biomarker values include a first biomarker value for a level of nitric oxide in the user, and wherein the thermal image pre-processing subsystem is configured to process the thermal image to determine the first biomarker value.

6. The system of claim 5 wherein the thermal image pre-processing subsystem is configured to process the thermal image to determine a measure of an area of the body part within a temperature range to determine the first biomarker value for the level of nitric oxide in the user.

7. The system of claim 1, wherein the one or more biomarker values include an audio biomarker value for a respiratory infection or respiratory disease in the user, and further comprising a microphone to capture a sound from the user, and wherein the infection or disease sensing system is further configured to process the sound to determine the audio biomarker value.

8. The system of claim 7 wherein the infection or disease sensing system includes a machine learning system trained to process the sound to determine the biomarker value for the respiratory infection or respiratory disease.

9. The system of claim 1, wherein the one or more biomarker values include a second biomarker value for a level of gas or nitric oxide in the user, and further comprising a gas or nitric oxide sensor to sense gas or nitric oxide from the user, and wherein the infection or disease sensing system is further configured to determine the second biomarker value.

10. (canceled)

11. The system of claim 1, wherein the one or more biomarker values include a biomarker value for a level of sweat on the surface of the body part, and further comprising a moisture sensor to sense moisture on a surface of the body part, and wherein the infection or disease sensing system is further configured to determine the biomarker value for a level of sweat on the surface of the body part.

12. The system of claim 11 wherein the moisture sensor comprises an optical reflectivity sensor to remotely sense moisture on the surface of the body part.

13. The system of claim 1, wherein the one or more biomarker values include a biomarker value for a point temperature at a target location on the surface of the body part, and further comprising a point temperature sensing system to remotely sense a temperature at a point location, and wherein the infection or disease sensing system is further configured to determine a biomarker value for the point temperature at a target location on the surface of the body part.

14. The system of claim 13 further comprising a system to detect a position of the target location in relation to a position of the point location, and to provide feedback to a user to enable the user to move the body part in relation to the point location so that the point location and target location coincide.

15. The system of claim 1, further comprising a system to identify the user and determine a user ID, and wherein the infection or disease sensing system is configured to determine, for storage and/or transmission, a cryptographically protected combination of the user ID and one or more of: the body temperature of the user, the one or more further characteristics, and data from the classification output.

16. The system of claim 15 wherein the cryptographically protected combination comprises a blockchain to link the user ID with a timestamped block comprising the one or more of: the body temperature of the user, the one or more further characteristics, and the data from the classification output.

17. The system of claim 1, wherein the body part comprises a wrist and/or forearm of the user.

18. The system of claim 1, wherein the plurality of infection or disease conditions comprise one or more of: no detected infection, a coronavirus infection, heart disease, asthma, and an inflammatory infection.

19.-21. (canceled)

22. A system for remote body temperature measurement of a person or animal, comprising:
a first imaging sensor to capture a first image of a body part of the person or animal;
a thermal imaging camera to capture a thermal image of the body part;
wherein the first imaging sensor and the thermal imaging camera each have a field of view which includes the body part;
a first image processor to process the first image to identify when the body part is present in a field of view of the thermal imaging camera;
a thermal image processor to process the thermal image to identify one or more blood vessels in the field of the thermal imaging camera;
wherein the system is configured to determine a body temperature of the person or animal from the thermal image of the blood vessels;
wherein the body temperature is determined in response to the identification of when the body part is present in a field of the thermal imaging camera.

23.-25. (canceled)

26. A system as claimed in claim 22 in combination with a radio frequency card or token reader, wherein the first imaging sensor and the thermal imaging camera have overlapping fields of view, and wherein the visual camera and the thermal imaging camera are located adjacent the card or token reader such that when the person's hand holds the card or token their wrist is located in the overlapping fields of view.

27. A system as claimed in claim 22, wherein the thermal image processor is further configured to process the thermal image to identify a pattern of blood vessels and/or bones in the wrist, and in response to determine an identifier for the person.

28.-37. (canceled)
